(19) United States
(12) Patent Application Publication
(10) Pub. No.: US 2002/0176849 A1
(43) Pub. Date: Nov. 28, 2002

(54) Endomural therapy

(75) Inventor: Marvin J. Slepian, Tucson, AZ (US)

Correspondence Address: Patrea L. Pabst, Holland & Knight LLP, One Atlantic Center, Suite 2000, 1201 West Peachtree Street, Atlanta, GA, 30309-3400, US

(73) Assignee: Endoluminal Therapeutics, Inc.

(21) Appl. No.: 10/072,766

(22) Filed: Feb. 8, 2002

Related U.S. Application Data
(60) Provisional application No. 60/267,578, filed on Feb. 9, 2001.

(51) Int. Cl. A61K 38/48; A61K 31/20; A61K 31/715; A61K 31/74; A61K 38/18

(52) U.S. Cl. 424/93.7; 424/78.08; 514/12; 514/54; 424/94.63; 514/560

(57) ABSTRACT

Methods, devices and materials for the treatment or repair, replacement, transplantation or augmentation of tissues in endomural zones specifically by open surgical, minimally invasive or percutaneous transmural or trans parenchymal application of polymeric material alone or in combination with bioactive agents or cells, have been developed. These methods and systems are useful to repair, alter function, replace function or augment function of the central or endomural aspects of solid organs or tubular body structures.

### BACKGROUND OF THE INVENTION

This invention relates to an aspect of in situ tissue engineering of organs or organ components, and repair, replacement, or alteration of function via manipulations targeted to the middle or endomural aspects of tissues.

This application claims priority to U.S. Ser. No. 60/267,578 filed Feb. 9, 2001.

This invention relates to devices, materials and methods for the treatment or repair of tissues, specifically by accessing the endomural zone (middle zone) of organs, organ components or tissues, either via surgical or percutaneous application of devices, polymeric materials, alone or in combination with bioactive agents or cells.

Many diseases involve the central aspects of organs, e.g. tumors in the liver, atherosclerotic lesions within the walls of arteries, adenomas in the prostate, malignancies within the brain, etc. Today the majority of these types of lesions are removed via open surgical, minimally invasive or percutaneous procedures which make direct incisions into the organ beginning on the ectoluminal or endoluminal surface. As such these approaches remove much healthy tissue in surrounding unaffected tissue layers, in the process of removing or treating the diseased zone. For example, in open surgical removal of an intra-organ tumor the capsule and ectoluminal zones as well as surrounding endomural healthy zones are often violated and excised in the process of removing the contained disease zone. While this therapy is effective it has the added morbidity burden of “mass” rather than “selective” destruction or treatment.

The same issue of unnecessary tissue damage and removal holds true for percutaneous or endoluminally accessed and treated disease. Access to the endoluminal regions via this route has traditionally removed the overlying endoluminal layers and surrounding endomural healthy zones to get at diseased zones within. An example of this may be seen in the current therapy for prostatic adenomas, the TURP (Transuretheral resection of the Prostate) procedure. In TURP the normal urothelial mucosal layer is removed as well as the peri-urethral column to gain access and remove intra-organ contained adenomas. This approach, while effective in removing the contained disease zone, unfortunately removes a significant amount of normal non-diseased tissue at the same time. As such, current procedures carry unnecessary morbidity and mortality due to their invasiveness and associated trauma.

Another limitation of current therapies lies in the fact that many diseases involve cellular derangement in the endomural zones while current therapies often only treat outer, either endoluminal or ectoluminal, zones. An example of this may be seen in therapy of atherosclertic lesions of coronary and peripheral arteries. In cases of severe atherosclerotic obstruction, endovascular removal of obstructive lesions via endovascular atherectomy, a catheter-based shaving, coring or drilling procedure from within the vessel is often employed. These approaches remove the diseased atheroma close to the vessel lumen and close to the treatment device. However, they do not tackle the source or “core” of the disease which frequently lies in the media of the artery, the endomural zone of the vessel.

Many therapies today are administered systemically with the goal of achieving a local intra-organ effect. If systems and methods existed which would provide mechanical physical targeting with simultaneous sustained intra-organ presence more effective, more accurate, site specific therapies would be achieved. Many “local” therapies are not local but regional and in fact affect adjacent zones. An example of this is intra-arterial chemotherapy for intra-hepatic malignancy such as hepatoma or hepatic metastasis. In this therapy drugs are adminstered via the hepatic arterial or vasculature system to treat disease within the organ but in fact the entire organ from within and without is bathed with medication. Further hepatically delivered medication subsequently diffuses or mixes directly intralumenally with systemic blood.

It is therefore an object of the present invention to provide methods and devices for treatment of diseased organs or tissues with minimal damage to surrounding tissue.

It is a further object of the present invention to provide methods and devices for treatment of cental, core or generally “endomural” zones of diseased organs or tissues with minimal damage to surrounding tissue

It is a further object of the present invention to provide methods and devices for treatment of specific tissues while avoiding systemic toxicity.

It is a further object of the present invention to provide polymeric materials, drugs and biologically active compositions which can be delivered or released endomurally to aid in healing.

It is a still further object of the present invention to provide devices, both surgical and percutaneous to access and modify endomural tissues and/or deliver polymeric materials, drugs and biologically active compositions which can be delivered or released endomurally to aid in healing.

### SUMMARY OF THE INVENTION

Methods, devices and materials for the treatment or repair, replacement, transplantation or augmentation of tissues in endomural zones specifically by open surgical, minimally invasive or percutaneous transmural or trans parenchymal application of polymeric material alone or in combination with bioactive agents or cells, have been developed. These methods and systems are useful to repair, alter function, replace function or augment function of the central or endomural aspects of solid organs or tubular body structures.

### BRIEF DESCRIPTION OF THE DRAWINGS

FIG. 1 is a diagram of a patient showing the various means of accessing the endomural zones: intra sinus, intrathecal, percutaneous transthoracic, percutaneous transabdominal, percutaneous—open, transarterial, transvenous, translymphatic, subcutaneous, and surgical.

FIGS. 2A-G are diagrams showing introduction of bioactive material using a catheter including a reservoir and control means to access the organ (A), where the catheter is positioned and stabilized (B), the endoluminal zone penetrated (C), stabilized in the endomural zone (D), optionally further including the step of removing tissue, using mechanical, laser, thermal, radiofrequency, ultraviolet, x-ray, electromagnetic, acoustic or chemical means (E), delivering biologically active agents, including pharamcologic agents, cells, or biomaterials (G), and sealing the zone and access tract (H).

FIGS. 3A and 3B a catheter including expansive means for delivery of drug particles (FIG. 3A) into the endomural zone (FIG. 3B).

FIGS. 4A and 4B are a catheter including an actuator means for expanding expansive means for delivery of drug particles into the endomural zone (FIG. 4A) and an expanded view of the actuator means (FIG. 4B).

FIGS. 5A and 5B are a catheter including a piezoelectric pump (FIG. 5a), where the catheter further includes a spray nozzle for dispensing drug particles into endomural tissue, guide means, and a reservoir for fluids or polymer and an optional heating element for melting of the polymer.

### DETAILED DESCRIPTION OF THE INVENTION

A method has been developed for treatment of endomural tissue. The method generally include placing a tubular tissue accessing device (needle, trocar, catheter) percutaneously into the organ. Tissue is removed in the organ. A flowable preformed or dessicated hydrogel or solid polymer plug is placed into the hole, filling the void and sealing the tissue tract. This method is useful in a variety of applications. For example, the endocardial (or epicardial) surface is accessesed via delivery device, the device is stabilized against the heart wall, the myocardium is penetrated to access the endomural zones, a space or void is created, and cells, polymers,drugs and genes or combination of these in varying sequences can delivered to the myocardium. The void mass or plug is then sealed in place.

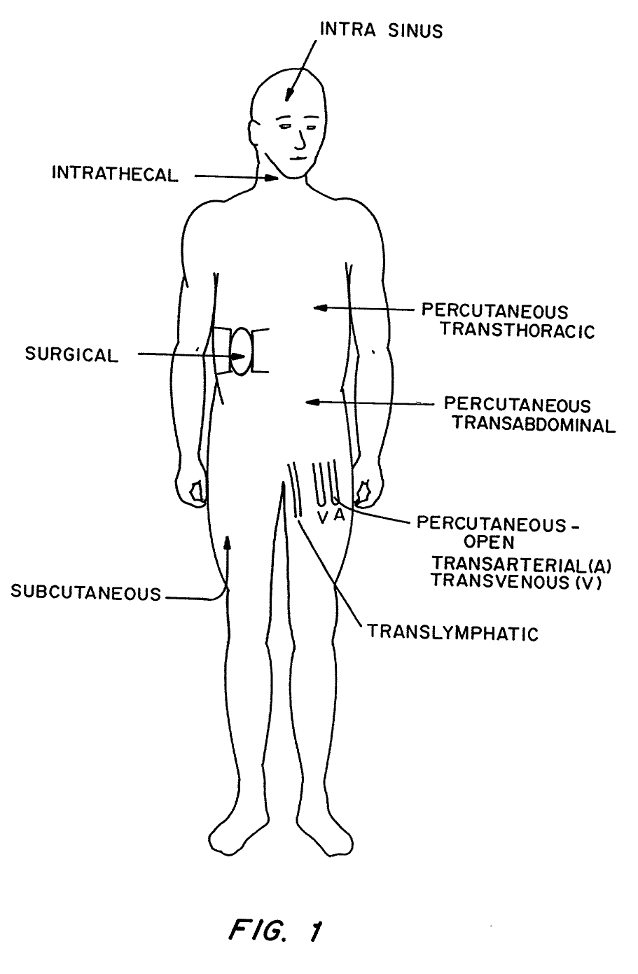

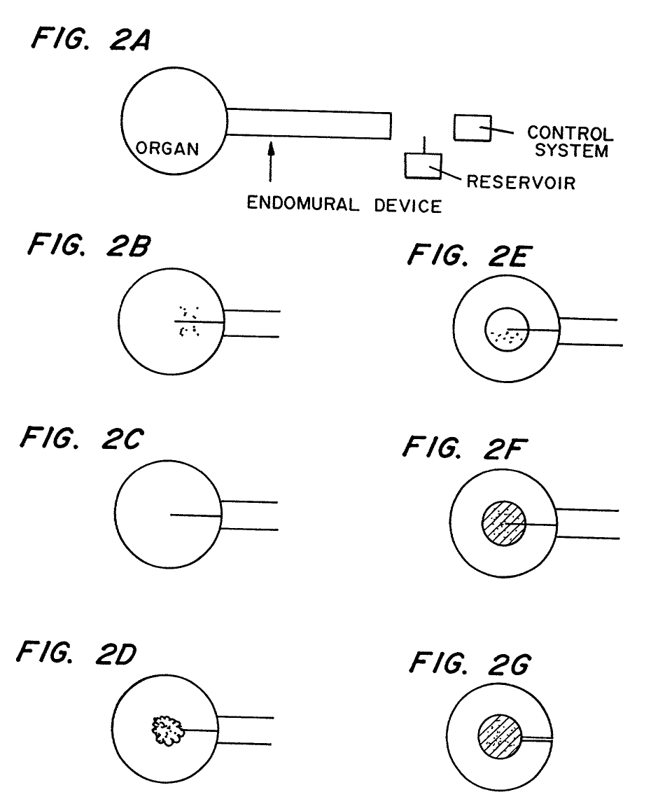

This method is generally shown in FIG. 1 and FIGS. 2A-2G. The method includes the steps of:

1. Access close to organ, either percutaneously, surgically, laparoscopically, transvacularly, transenterally, intrathecally, subcutaneously via tissue planes, translyphatically,etc.

2. Park and stabilize in location the delivery means.

3. For endo or ecto access, penetrate endoluminal zone, stabilize in endomural zone, locally treat tissue, and remove tissue—mechanically, thermally, with laser, radiofrequency, ultraviolet, x-ray (any form of tissue damaging), electromagnetic energy, acoustic energy (ultrasound), dessication, gas exposure (CO₂, ether), chemically—antimetabolics, antineoplastic, anti-inflammatory, antimicrobial, antiviral, antibiotics, hormones, antibodies, etc.

4. Deliver agents, such as pharmacologic agents, cells, or other biologicals or biomaterials.

5. Seal zone and access tract.

### Definitions

I. General Organization of Higher Animals:

The structural organization of higher animals such as mammals, including man, is that of multiple integrated and interactive tissue components. These tissues may be organized as discrete organs which are functional factories, e.g. liver producing biochemical mediators or device systems, e.g. heart—mechanically pumping blood and brain—electrically signaling and coordinating events. As referred to herein, organs include solid and hollow organs, e.g. the liver and colon, respectively.

Alternatively, animals contain tissue components which are largely conduits for functional fluids such as blood, lymph, endocrine or exocrine secretions or gases. These tubular “organ components” or conduits are structures such as arteries, veins, lymphatics, bile ducts, ureters, fallopian tubes, etc.

II. Structure of Organs and Organ Components—The Endomural Zone Defined

Discrete organs may be generically described as having three regions or zones. These regions include: 1. the ectoluminal or outer zone (i.e. capsule, serosa, etc.), 2. the endomural or middle zone and 3. the endoluminal zone. In discrete organs the ectoluminal region typically functions to protect and contain the organ. The endomural zone of the organ is typically the functional or “business end,” of the organ, acting as a biochemical factory for production of homeostatic proteins, hormones, enzymes and immunoglobulins for defense and reparative cells for tissue repair, organ regeneration,, metabolism or other specialized functions. In mechano-dynamic organs such as the heart and lung, the endomural zones function to propel or exchange fluid or gas. The inner or ectoluminal zone of organs may have functions similar to the endomural zone or act as yet another internal boundary or barrier layer. If an organ is cut in cross-section the ectoluminal zone may be characterized as the outer 10% ±10 cross-sectional area, the endomural zone as the mid 80% ±10 and the endoluminal zone as the inner 10% ±10.

In addition to solid or hollow organs with cavities true tubular organs and organ components exist as vital body structures. Examples of tubular organs include the small intestine and the colon. Tubular organ components include major interpenetrating blood vessels in organs, e.g. the portal vein in the liver, the cavernous sinus in the brain. Examples of tubular tissue structures, include ducts, e.g. the bile duct, or blood vessels, e.g., arteries or veins.

Tubular organs and tissue structures in general have a laminated, multilayer “tube-in-tube” structure made of at least three layers. All of these tubular organs, organ components or tissue structures may be characterized in similar fashion as outlined for organs above into ectoluminal, endomural and endoluminal zones. In tubular structures the ectoluminal zone may be characterized as the outer 10% ±10 cross-sectional area, the endomural zone as the mid 80% ±10 and the endoluminal zone as the inner 10% ±10. Interestingly, tubular organs and tissue structures have defined histologic layers which generally correlate with these zones. The ectoluminal zone correlates with serosa or adventitia. The endoluminal zone correlates with the lamina propria, submucosa, muscularis, or media. The endoluminal zone correlates with the intima or mucosa.

### Methods of Treatment

I. Localized Treatment

Methods which focus on treatment of the endomural region of an organ or tissue provide a means to reduce the trauma to adjacent, contiguous or “collateral,” healthy tissue associated with removing, containing or locally treating active disease within central or endomural regions of an organ or tissue structure. This also allows the disease to be treated more effectively, on a local basis, with agents, cells or systems without risk of systemic exposure. Through local application of polymers, pharmaceuticals, genes, therapeutic peptides, cells, radiation systems, etc., one is able to focus therapy to the affected zone of an organ while sparing exposure of surrounding contiguous or adjacent healthy tissue. Local intra-organ therapy reduces systemic exposure to agents which may have deleterious effects systemically. This allows application of higher effective concentration of agents without fear of toxicities with reduced systemic spillover effects.

Endomural treatment also provides a means for sustained durable local therapy, as well as containment and hence sustained exposure or therapeutic presence in an organ compared with conventional parenteral or topical therapy, over longer periods of time than are typical with systemic delivery. Creating cavities or pockets within an organ allows “rebuilding” and reconstruction from inside. Placing therapeutic agents or materials in a “privileged zone, ” free from overlying blood flow, increases retention and thereby sustains action of the agents. This also provides for more accurate therapy.

Endomural treatment not only localizes the treatment modalities, but also cordons off disease physically, creating barriers to the disease as well as local treatment of the disease.

II. Use of Endomural regions of Organs as Seed Beds

Endomural therapy provides the potential to utilize one organ bed or body as a soil in which to place or plant cells, or organ components to provide another function normally provided by another organ. In many disease states a vital function of an organ is diminished or destroyed by a disease. Conventional therapy aims to pharmacologically limit resultant symptoms or attempt to restore lost function. These approaches are limited. Despite disease of the organ, the remaining tissue components or stroma often have relevant functions themselves. Further, even if specialized tissues and cells of a given organ are diseased, the vascular, neural and stromal matrix of the organ are often intact and are a functional generic organ bed. These residual structures may be looked upon as a fertile “soil” for transplantation or implantation of cells, cell-polymer combinations, other organ components, organoids, artificial organs or bioreactors. These diseased organ shells will function to provide a bed for engraftment of these implants with “housekeeping functions, ” i.e. arterial and venous supply, lymphatic drainage, innervation, etc., already built-in and intact.

III. Application of Polymeric Structural or Bioactive Materials

As noted above, therapeutic materials such as drugs and cells can be administered and contained intramurally, for treatment of a disease or to provide supplementary function. Other materials, for example, polymers having additional properties such as the ability to facilitate healing, minimize or provoke inflammation, decrease fibrotic response, inhibit abnormal proliferation or other therapeutic benefits, may also be utilized. Polymers may be themselves bioactive or contain embedded or grafted bioactive molecules, peptides, lipids, drugs or other moieties. These polymers may either suppress, maintain or stimulate a biological response. The polymers may also serve as tissue glues, adhesives or sealants to isolate tissue zones, creating internal barriers. These polymers may also serve to provide an artificial biodegradable or permanent scaffold or stroma for implanted or transplanted cells, fragments or tissues.

IV. How to access organ

An organ or tissue can be accessed surgically, either by open exposure or using minimally invasive techniques; or percutaneously.

The endomural regions of an organ or tissue can also be accessed surgically, through open exposure of internal organs or though trans-body wall incisions. This is typically followed by defined focused narrow puncture of the organ, without open radical dissection, and subsequent entry into the endomural zone and placement of a therapeutic.

Endoluminal entry is typically achieved via the use of needles, trocars, ballistic transfer—explosive bullet-like, spark projection, projectile pellets e.g. gene gun, pneumatic transfer (high pressure air, CO₂), chemical permeation, optical or other irradiation-based penetration, ultrasound, electroporation or pheresis—mediated transfer.

These routes and means of penetration are minimally invasive, may be used via direct tissue contact or through key-hole or other limited port entry into the inner aspects of the body, with subsequent defined focused contact and similar penetration means or through subsequent narrow or limited physical puncture of the organ, without open radical dissection, and subsequent entry into the endomural zone for placement of a therapeutic either directly or through the above limited penetration, permeation or other transport means.

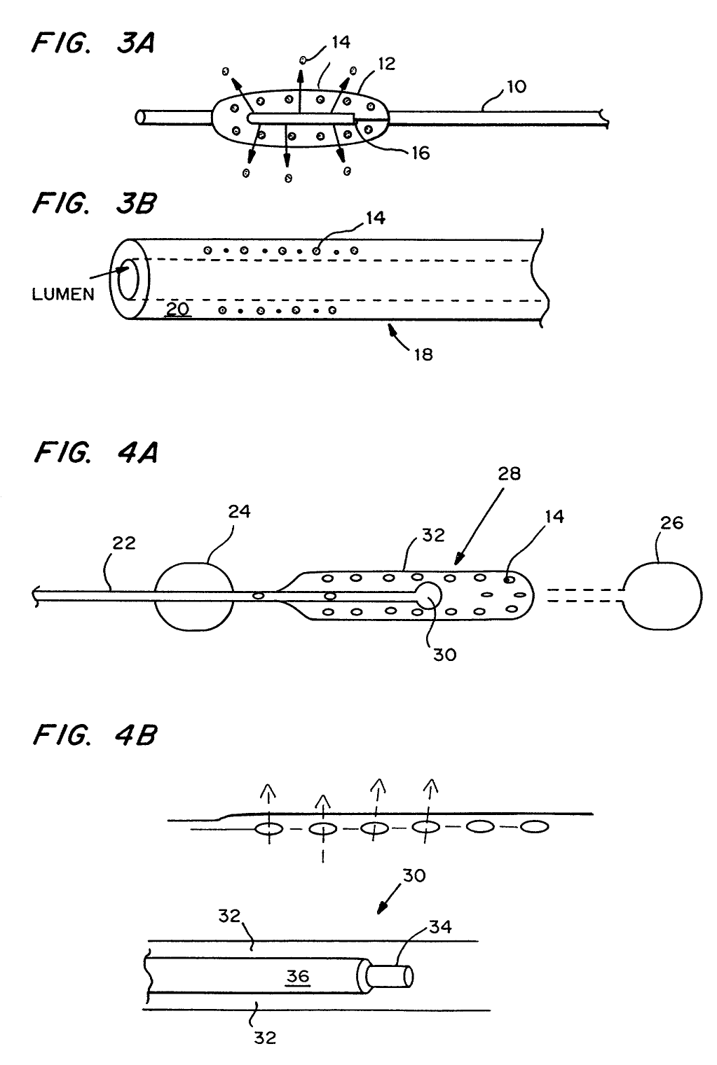

FIGS. 3-5 demonstrate devices which may be used for this purpose. FIG. 3A shows a simple balloon device, wherein the catheter 10 includes a balloon 12 permeable to the drug particles 14 to be delivered. An activating or propelling agent or other means 16 within the balloon 12 is used to propel the drug particles 14 out of the balloon 12 and into the tissue as shown in FIG. 3B. FIG. 3B shows a blood vessel 18 wherein the drug particles 14 have become embedded within the endomural zone 20.

In another embodiment shown in FIG. 4A, drug particles 14 can be delivered to a desired location within the endomural zone by introducing a catheter 22 into the tissue lumen, wherein the catheter22 has two expansile members 24 and 26, typically balloons, and means 28 for delivering the drug particles 14 at a space between the two members 24 and 26; expanding the expansile members 24 and 26 to occlude the targeted portion of the lumen, administering the drug particles 14 by administering a force via an actuator means 30 that propels the drug particles 14 through a macroporous membrane 32 and into the endomural zone, contracting the expansile members 24 and 26, and removing the catheter 22. In one preferred embodiment, the catheter is also used to wash out the occluded region so that, in the case of a blood vessel, the region is substantially free of blood. FIG. 4B is an expanded view of the actuator means 30, with expandable walls 32, a tip 34 to insert the actuator means into the delivery means 28, and propellent means 36. The propellent means 36 can be an explosive, hydraulic, or other energy generating means.

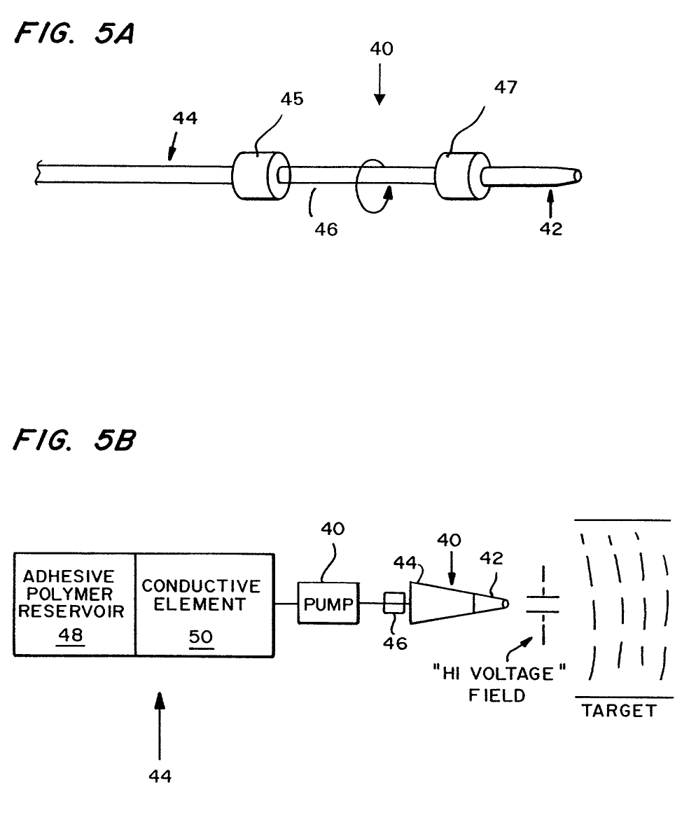

As shown in FIG. 5A, the drug particles can be delivered using other means, such as a piezoelectric pump 40. The pump 40 includes a nozzle 42 which is rotatable as well as capable of being angled to deliver drug to the appropriate target. This is attached to a catheter 44 including a proximal balloon, distal balloon, guide wire (or other steering means) 46, and, optionally, means 48 for dispersing one or more other materials (including washing or irrigation fluids, adhesive or polymer solutions), etc. and optionally conductive means for heating materials 50, as shown in FIG. 5B.

Delivery can also be via a percutaneous route, for example, through transcutaneous entry into conduit systems or “highways” of the body. One advances to the desired region of interest under direct visual guidance, fluoroscopy or ultrasonic guidance, with subsequent entry into the endomural and/or endoluminal zones and placement of a therapeutic, as necessary as outlined above.

Implantable devices or delivery means can include sensors for data measurement, and/or data analyzers, and/or data storage means, and/or data telemetry/transmission means including means for communication at mutiple levels of isolated or nested levels of information transfer. These devices may have incorporated means for modification of the implant or mounting a response, e.g. local or systemic drug delivery, in response to measurements made using the sensors. These are particularly useful in urology, hepatology or cardiology, where the implants contain one or more sensors responsive to variables which change over time, for example, pressure which is indicative of changes in fluid flow and diameter of the ureter, biliary duct or vessel in which the implant has been placed. Feedback from the sensor(s) either directly, or indirectly via monitoring means external to the patient, signal changes that may be required, such as expansion of the implant in the case where the tissue lumen diameter changes over time or the implant becomes unstable or migrates. In another embodiment, the implant contains a bioactive, prophylactic, diagnostic or pH modifying agent. In one embodiment, the implant is formed of a temperature or pH responsive material so that the agent is released when the temperature or pH is altered.

These systems can also be used to connect a patient to a remote data storage or manipulation system, such as a watch-like device, small portable device, intra or extradermal implant, phone system devices (portable phones, answering services, beepers, office fax machines), portable computer, personal digital assistant (PDA, e.g., Palm Pilot™ systems), or to the internet (world wide web) or a computer accessible through devices that the physician or nurse can monitor or use to interact remotely with the implant.

V. How to create repository zones in organ

Voids may be created via simple catheter, trochar or needle insertion. The void may be of identical size to the insertion device. Alternatively, the void may be made larger via expansile cutter systems which fan-out in a radial or conical or other geometric shape way. Voids may also be created via other mechanical means, e.g. tissue morcellator, balloon dilator, mechanical tissue jack or stretcher, thermal, electrical, ultrasonic laser, UV, x-ray, or other injurious or ablative electromagnetic radiation, cryogenic, chemical—e.g. acids. alkali, detergents, osmotic fragility means, or enzymatic means, e. g. papain, trypsin, chymotrypsin, matrix metalloproteinases, fibrolytic agents, streptokinase, and tissue plasminogen activator. Aspiration, perfusion or superfusion may be used to further wash and expand the voids.

Voids may be filled with drugs, polymers, polymer-drug mixtures or covalently linked drug-polymer combinations. Polymers may be utilized to further facilitate void creation via delivery of void forming agents, to fill an initially created void for therapeutic purposes, to deliver subsequent therapeutic agents in a tiered or sequenced therapeutic scheme, to limit further void expansion, to provide a neomatrix or scaffold for subsequent cell or tissue engraftment or to form a void- or cavity-barrier limiting void entry or exit. Further, these barriers may be selectively permeable in either a unidirectional or bi-directional fashion.

Polymers may be therapeutic or serve as the means for delivering therapeutic agents. Polymers may be inserted in simple spaces created via device insertion or in larger spaces created as a result of initially creating tissue defects, voids or other cavities. Voids created as a result of disease, defect or surgical procedure are filled with adhesive polymers that facilitate void cavity wall bonding and healing. Polymers are specifically selected to minimize inflammation, secondary bleeding and late fibrotic scarring. Alternatively if an angiogenic or fibrogenic response is desired, polymers may be selected so as to induce a pro-inflammatory, angiogenic, fibrogenic response.

Tissue voids within an organ can be filled with biocompatible biodegradable polymers to act as intra-void tissue bonding agents, allowing collapse and exclusion of the void space while simultaneously increasing intramural lumen space. The polymers may either spontaneously solidify or they may be polymerized or bound to the tissue upon exposure to an appropriate stimulus, as discussed in more detail below. Polymer may posses “therapeutic” hygroscopic or hydrophobic properties to either facilitate progressive water uptake and void shrinkage or to prevent uptake allowing tissue swelling. The polymers are selected to facilitate healing, with minimal inflammatory and late fibrotic responses. Coordinating use of tissue friendly biodegradable polymeric bioadhesives insures frank volume reduction and obliteration of cavities formed via direct tissue excision. Furthermore, the polymeric materials having drugs, genes or cells incorporated therein may serve as local depots for prolonged delivery of synergistic biochemical and cellular therapeutics, for example, to promote healing, decrease inflammation and/or collagen deposition and scarring, and manipulate endocrine processes and local growth control.

VI. How to implant in the organ

These materials can be implanted in the organ directly, in repository zones, created as described above. Materials to be implanted other than drugs and polymers include cells. Cells can be grown in vitro, in cell culture or obtained by biopsy. Cells may be genetically modified. Cells may be isogeneic, allogenic or xenogeneic. Allogenic or xenogeneic cells may be encapsulated for immunotolerance.

Cells may be added as single cells, slurries of single or multiple cell types or from multiple sources, organ fragments or tissue shards. Cells added to a given organ or organ component may be identical or similar differentiated normal cells, different differentiated normal cells, progenitor cells, genetically transfected, transformed or engineered cells, stem cells, embryonic cells, multipotential cells, primordial cells, allogeneic, heterogeneic, xenograft cells, encapsulated allogeneic, heterogeneic, or xenograft cells. Therapeutic non-mammalian, eukaryotic, plant or prokaryotic cells may be delivered.

Therapeutic biologicals such as cell fragments, heterokaryons, viruses, pseudovirions, viroids, prions, DNA, or RNA (sense, antisense, ribozymes or aptemers) may be co-delivered.

Plant cells, prokaryotic cells, or artificial cells may be administered as therapeutically indicated as well. These cells may be passivated or encapsulated to facilitate seeding and routing and to prevent immunorejection.

Cells or tissues from different organs may be transplanted from one organ to function as a substitute in another organ. For example, one could transplant splenocytes into a liver shell or scar or myocardial scar to act as angiogenic precursors. One could transplant neural stem cells or dorsal root ganglion cells into the heart of patients with diabetes to return sensation of angina as a therapeutically beneficial return of a clinical warning sign. One could transplant splenocytes into bone marow to act as hematologic precursors.

VI. Polymeric or Hydrogel Materials

Biodegradable and/or biocompatible materials may be used to fill, shape, bulk or adhere to voids, cavities, channels or other spaces created by the endomural therapeutic devices to enhance healing, to provide structural support within the cavity, tubular organ or organ component a to assist or obviate the need for other lumen or cavity support following surgery, and/or for drug delivery. For example, polymeric or hydrogel materials can be applied at the surface of or interior of cavities created by removal of tissue to treat the disorders caused by overproliferation or inflammation of tissue. These materials can be used to adhere the sides of the tissue cavity together, to form a barrier at the surface of one or more of the tissue surfaces (to minimize inflammatory processes, for example), for delivery of bioactive agents, for the retention of radioisotopes, radioopaque particulate etc. The polymer may be deployed in the interior of the endomural tissue of the vessel or organ from the surface or tip of the catheter, as discussed above. Alternatively, the polymer can be applied by spraying, extruding or otherwise internally delivered via a long flexible tubular device consisting of as many lumens as a particular application may dictate.

Preferably, the method utilizes biodegradable or bioerodible synthetic or natural polymers, with specific degradation, lifespan and properties, which can be applied in custom designs, with varying thicknesses, lengths, and three-dimensional geometries (e.g. spot, stellate, linear, cylindrical, arcuate, spiral 8, etc.). The pharmaceutical delivery function of the process may be readily combined with the “customizable” deployment geometry capabilities to accommodate the interior of a myriad of complex organ or vessel surfaces. For example, polymer can be applied in either single or multiple polymer layer configurations and different pharmacological agents can be administered by application in different polymer layers when multiple polymer layers are used.

1. Selection of Polymeric Materials

A variety of different materials can be used, depending on the purpose, for example, structural, adhesive, barrier, or drug delivery. For those applications where structure is required, a polymer is selected which has appropriate mechanical and physical properties. It is preferred that the polymer be biodegradable over a period of time required to heal and form the tissue as desired according to the application. This may be a few days, weeks, or months. An advantage of the polymeric materials is that they can be tailored to shape the polymer into uneven surface interstices, while maintaining a smooth surface with good flow or other tissue compatibility characteristics. Tissue narrowing, if it does occur, tends to stabilize beyond the six month window following the initial procedure without further accelerated narrowing. Optimally, if a foreign support device or sealant material is to be introduced into the tissue, it needs to exert its intended effect principally during the period of healing and peak inflammatory reaction. Although described herein principally with reference to polymeric materials, it is to be understood that other materials may also be used. For example, relatively low molecular weight organic compounds such as common sugars (e.g. sucrose), which are cast from concentrated, warm aqueous solution to set up as monolithic solids in situ and erode with minimal swelling or fragmentation may be used in place of a polymeric material. Inorganic compounds formed by ion exchange, such as polysilicic acid salts, degradable bioceramics, and “plasters” which degrade by surface erosion but which set in situ can also be used.

For those applications where the purpose does not require structural support properties, the polymer may be formed of a material that is bioadhesive, or impermeable to molecules of specified molecular weights, or highly permeable, releasing incorporating drug over a desired period of time, and consist of as little as a single layer of polymer.

Accordingly, the nature of the polymeric material used will be determined by whether it functions as a coating, bandage, adhesive, drug delivery device, or mechanical support role. Further, the choice of polymer must appropriately balance the degree of structural and geometric integrity needed against the appropriate rate of biodegradation over the time period targeted to prevent an undesirable reaction. In some cases, the material may be the same for different purposes where the ultimate in vivo geometry of the polymer dictates the final function of the polymer coating. The thinner applications allow the polymer film to function as a coating, sealant and/or partitioning barrier, bandage, and drug depot. Complex internal applications of thicker layers of polymer may actually provide increased structural support and, depending on the amount of polymer used in the layer, may actually serve in a mechanical role to maintain vessel or organ patency. For example, lesions of tissues that are comprised mostly of fibromuscular components have a high degree of visco-elastic recoil. These lesions or tissues require using the process to apply an endomural coating of greater thickness or stiffness and extent so as to impart more structural stability thereby resisting vessel radial compressive forces. This provides structural stability and is generally applicable for the maintenance of the intraluminal geometry of all tubular biological organs or substructure.

The basic requirements for the polymeric material are biocompatibility and the capacity to be applied in a solid or fluent state then chemically or physically reconfigured under conditions which can be achieved in vivo to yield a non-fluent polymeric material having defined characteristics in terms of mechanical strength, permeability, adhesion, and/or release of incorporated materials.

The polymeric materials can be applied as polymers, monomers, macromers or combinations thereof, maintained as solutions, suspensions, or dispersions, referred to herein jointly as “solutions” unless otherwise stated. Polymeric materials can be thermosettable, thermoplastic, polymerizable in response to free radical or ioin formation such as by photopolymerization, chemically or ionically crosslinkable (i.e., through the use of agents such as glutaraldehyde or ions like calcium ions). Examples of means of solidifying or polymerizing the polymeric materials including application of exogenous means, for example, application of light, ultrasound, radiation, or chelation, alone or in the presence of added catalyst, or by endogenous means, for example, a change to physiological pH, diffusion of calcium ions (e.g., alginate) or borate ions (e.g., polyvinyl alcohol) into the polymeric material, or change in temperature to body temperature (37° C.).

Although either non-biodegradable or biodegradable materials can be used, biodegradable materials are preferred. As used herein, “biodegradable” is intended to describe materials that are broken down into smaller units by hydrolysis, oxidative cleavage or enzymatic action under in vivo conditions, over a period typically less than one year, more typically less than a few months or weeks. For application to tissues to prevent inflammation, enlargement and/or overproliferation, it is preferred to use polymers degrading substantially within six months after implantation. For prevention of adhesions or controlled release, the time over which degradation occurs should be correlated with the time required for healing, i.e., generally in excess of two weeks but less than six months.

Suitable materials are commercially available or readily synthesizable using methods known to those skilled in the art. These materials include: soluble and insoluble, biodegradable and nonbiodegradable natural or synthetic polymers. These can be hydrogels or thermoplastics, homopolymers, copolymers or blends, natural or synthetic. As used herein, a hydrogel is defined as an aqueous phase with an interlaced polymeric component, preferably with 90% of its weight as water. The following definition is from the Dictionary of Chemical Terms, 4th Ed., McGraw Hill (1989): Hydrogel: a colloid in which the disperse phase (colloid) has combined with the continuous phase (water) to produce a viscous jellylike product, for example, coagulated silicic acid. An organogel is defined as an organic phase with an interlaced polymeric component, preferably with 90% of its weight as organic solvent. Preferred solvents include non-toxic organic solvents, such as dimethyl sulfoxide (DMSO), and mineral and vegetable oils. The preferred polymers are synthetic polymers, formable or synthesizable in situ, with controlled synthesis and degradation characteristics.

Representative natural polymers include proteins, such as zein, modified zein, casein, gelatin, gluten, serum albumin, or collagen, and polysaccharides, such as cellulose, dextrans, hyaluronic acid, polymers of acrylic and methacrylic esters and alginic acid. These are not preferred due to higher levels of variability in the characteristics of the final products, as well as in degradation following administration. Synthetically modified natural polymers include alkyl celluloses, hydroxyalkyl celluloses, cellulose ethers, cellulose esters, and nitrocelluloses, acrylic or methacrylic esters of above natural polymers to introduce unsaturation into the biopolymers.

Representative synthetic polymers include polyesters, polyphosphazines, poly(vinyl alcohols), polyamides, polycarbonates, polyalkylenes, polyacrylamides, polyalkylene glycols, polyalkylene oxides, polyalkylene terephthalates, polyvinyl ethers, polyvinyl esters, polyvinyl halides, polyvinylpyrrolidone, polysiloxanes, polyurethanes and copolymers thereof. Other polymers include celluloses such as methyl cellulose, ethyl cellulose, hydroxypropyl cellulose, hydroxypropyl methyl cellulose, hydroxybutyl methyl cellulose, cellulose acetate, cellulose propionate, cellulose acetate butyrate, cellulose acetate phthalate, carboxymethyl cellulose, cellulose triacetate, cellulose sulfate sodium salt, acrylates such as poly(methyl methacrylate), poly(ethyl methacrylate), poly(butyl methacrylate), poly(hexyl methacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate), polyethylene, polypropylene, poly(ethylene glycol), poly(ethylene oxide), poly(vinyl acetate), polyvinyl chloride, polystyrene, polyvinyl pyrrolidone, and polyvinylphenol. Representative bioerodible polymers include polylactides, polyglycolides and copolymers thereof, poly(hydroxy butyric acid), poly(hydroxyvaleric acid), poly(lactide-co-caprolactone), poly[lactide-co-glycolide], polyanhydrides, polyorthoesters, blends and copolymers thereof.

These polymers can be obtained from sources such as Sigma Chemical Co., St. Louis, Mo., Polysciences, Warrenton, Pa., Aldrich, Milwaukee, Wis., Fluka, Ronkonkoma, N.Y., and BioRad, Richmond, Calif. or else synthesized from monomers obtained from these suppliers using standard techniques.

These materials can be further categorized as follows. Materials which polymerize or alter viscosity as a function of temperature.

Poly(oxyalkene) polymers and copolymers such as poly(ethylene oxide)-poly(propylene oxide) (PEO-PPO) copolymers, and copolymers and blends of these polymers with polymers such as poly(alpha-hydroxy acids), including but not limited to lactic, glycolic and hydroxybutyric acids, polycaprolactones, and polyvalerolactones, can be synthesized or commercially obtained. For example, polyoxyalkylene copolymers are described by U.S. Pat. Nos. 3,829,506; 3,535,307; 3,036,118; 2,979,578; 2,677,700; and 2,675,619, the teachings of which are incorporated herein. Polyoxyalkylene copolymers are sold by BASF and others under the tradename Pluronic™. Preferred materials include F-127, F-108, and for mixtures with other gel materials, F-67. These materials are applied as viscous solutions at room temperature or lower which solidify at the higher body temperature. Another example is a low Tm and low Tg grade of styrene-butadiene-styrene block copolymer from Polymer Concept Technologies, C-flex™. Polymer solutions that are liquid at an elevated temperature but solid at body temperature can also be utilized. For example, thermosetting biodegradable polymers for in vivo use are described in U.S. Pat. No. 4,938,763 to Dunn, et al.

Several divalent ions including calcium, barium, magnesium, copper, and iron are normal constitutents of the body tissues and blood. These ions can be used to ionically crosslink polymers such as the naturally occurring polymers collagen, fibrin, elastin, agarose, agar, polysaccharides such as hyaluronic acid, hyalobiuronic acid, heparin, cellulose, alginate, curdlan, chitin, and chitosan, and derivatives thereof cellulose acetate, carboxymethyl cellulose, hydroxymethyl cellulose, cellulose sulfate sodium salt, and ethylcellulose. Materials that can be crosslinked photochemically, with ultrasound or with radiation.

Materials that can be crosslinked using light, ultrasound or radiation will generally be those materials which contain a double bond or triple bond, preferably with an electron withdrawing substituent attached to the double or triple bond. Examples of suitable materials include the monomers which are polymerized into poly(acrylic acids) (i.e., Carbopols.™.), poly(acrylates), polyacrylamides, polyvinyl alcohols, acrylated polyethylene glycols, and ethylene vinyl acetates. Photopolymerization requires the presence of a photosensitizer, photoinitiator or both, any substance that either increases the rate of photoinitiated polymerization or shifts the wavelength at which polymerization occurs. The radiolysis of olefinic monomers results in the formation of cations, anions, and free radicals, all of which initiate chain polymerization, grafting and crosslinking and can be used to polymerize the same monomers as with photopolymerization. Photopolymerization can also be triggered by applying appropriate wavelength to a cyclo-dimerizable systems such as Coumarin and Cinnamic acid derivatives. Alpha-hydroxy acids backbone can be activated to carbonium ion. COOH or SO₃H functionality can be inserted that can be subsequently reacted to amine containing ligands Materials that can be crosslinked by addition of covalent crosslinking agents such as glutaraldehyde.

Any amino containing polymer can be covalently crosslinked using a dialdehyde such as glutaraldehyde, or succindialdehyde. Examples of useful amino containing polymers include polypeptides and proteins such as albumin, and polyethyleneimine. Peptides having specialized function, as described below, can also be covalently bound to these materials, for example, using crosslinking agents, during polymerization.

Polymers with free carboxylic acid or other anionic groups (e.g., sulfonic acid), such as the acrylic acid polymers noted above, can be used alone or added to other polymeric formulations to enhance tissue adhesiveness. Alternatively, materials that have tissue binding properties can be added to or bound to the polymeric material. Peptides with tissue adhesion properties are discussed below. Lectins that can be covalently attached to a polymeric material to render it target specific to the mucin and mucosal cell layer could be used. Useful lectin ligands include lectins isolated from: Abrus precatroius, Agaricus bisporus, Anguilla anguilla, Arachis hypogaea, Pandeiraea simplicifolia, Bauhinia purpurea, Caragan arobreseens, Cicer arietinum, Codium fragile, Datura stramonium, Dolichos biflorus, Erythrina corallodendron, Erythrina cristagalli, Euonymus europaeus, Glycine max, Helix aspersa, Helix pomatia, Lathyrus odoratus, Lens culinaris, Limulus polyphemus, Lysopersicon esculentum, Maclura pomifera, Momordica charantia, Mycoplasma gallisepticum, Naja mocambique, as well as the lectins Concanavalin A, Succinyl-Concanavalin A, Triticum vulgaris, Ulex europaeus I, II and III, Sambucus nigra, Maackia amurensis, Limax fluvus, Homarus americanus, Cancer antennarius, and Lotus tetragonolobus.

The attachment of any positively charged ligand, such as polyethyleneimine, polylysine or chitosan to any microsphere or polymeric chain may improve bioadhesion due to the electrostatic attraction of the cationic groups to the net negative charge of the mucus. A surfactant-like molecule bearing positive charge and a hydrophobic core would be compatible with the bilayer membrane. This molecule will distribute its core and the positive charge to minimize energy of interaction and hence will be more tissue adhesive. The mucopolysaccharides and mucoproteins of the mucin layer, especially the sialic acid residues, are responsible for the negatively charged surface layer. Any ligand with a high binding affinity for mucin could also be covalently linked to the polymeric material.

Polymeric materials can also be used as tissue adhesives. In one form, fibrin is used. This has the advantage that it can be formed easily in situ using the patient's own fibrinogen, blood or serum, by addition of thrombin and calcium chloride. The materials described above can also be used. Other polymeric tissue adhesives that are commercially available include cyanoacrylate glues, GRF (Gelatin-resorcinol-formaldehyde) and polyethyleneglycol-poly(lactic acid and/or glycolic acid)-acrylates, both of which are applied as liquids and then photopolymerized.

The polymeric material can be designed to achieve a controlled permeability, either for control of materials within the cavity or into the tissue or for release of incorporated materials. There are basically three situations that the polymeric material is designed to achieve with respect to materials present in the lumen: wherein there is essentially passage of only nutrients (small molecular weight compounds) and gases from the lumen through the polymeric material to the tissue lumen surface; wherein there is passage of nutrients, gases and macromolecules, including large proteins and most peptides; and wherein there is passage of nutrients, gases, macromolecules and cells. The molecular weight ranges of these materials are known and can therefore be used to calculate the desired porosity. For example, a macromolecule can be defined as having a molecular weight of greater than 1000 daltons; cells generally range from 600-700 nm to 10 microns, with aggregates of 30-40 microns in size. For passage of cell, the material must possess or develop a macroporous structure.

Formation of Materials Which Have Decreased Volume Following Polymerization

Under certain circumstances it may be useful to produce a polymer in situ which occupies a smaller volume than the solution from which it is applied, for example, as an adhesive for the cavity to hold the walls together. The polymerization can be accompanied by “syneresis” or expulsion of water from the polymer, during polymerization. Besides reducing mass of the product, this process may yield porous products that may be desirable for healing. Syneresis occurs when a polymerization reaction occurs with reaction of a large number of fractional groups per unit volume (high crosslinking density or when dilute solutions of reactants are polymerized and the amount of water in the formulation exceeds the intrinsic swelling capacity of the resulting polymer. The latter may occur, for example, when dilute solutions of PEG-diacrylate are polymerized (e.g., less than or equal to 5% macromer).

VII. Incorporation of Bioactive Agents

A wide variety of bioactive agents can be incorporated into the polymeric material. These can be physically incorporated or chemically incorporated into the polymeric material. Release of the physically incorporated material is achieved by diffusion and/or degradation of the polymeric material; release of the chemically incorporated material is achieved by degradation of the polymer or of a chemical link coupling the bioactive material to the polymer, for example, a peptide which is cleaved in vivo by an enzyme such as trypsin, thrombin or collagenase. In some cases, it may be desirable for the bioactive agent to remain associated with the polymeric material permanently or for an extended period, until after the polymeric material has degraded and removed from the site.

In the broadest sense, the bioactive materials can include proteins (as defined herein, including peptides generally construed to consist of less than 100 amino acids unless otherwise specified), saccharides, polysaccharides and carbohydrates, nucleic acids, and synthetic organic and inorganic materials, or combinations thereof.

Specific materials include antibiotics, antivirals, antiinflammatories, both steroidal and non-steroidal, antineoplastics, anti-spasmodics including channel blockers, modulators of cell-extracellular matrix interactions including cell growth inhibitors and anti-adhesion molecules, enzymes and enzyme inhibitors, anticoagulants, growth factors, DNA, RNA antisense, ribozymes, aptamers, and protein synthesis inhibitors, anti-cell migratory agents, anti-proliferative agents, vasodilating agents, and other drugs commonly used for the treatment of injury to tissue. Examples of these compounds include angiotensin converting enzyme inhibitors, anti-thrombotic agents, prostacyclin, heparin, salicylates, thrombolytic agents, anti-proliferative agents, nitrates, calcium channel blocking drugs, streptokinase, urokinase, tissue plasminogen activator (TPA) and anisoylated plasminogen activator (TPA) and anisoylated plasminogen-streptokinase activator complex (APSAC), GPIIb/IIIA antagonists, colchicine and alkylating agents, growth modulating factors such as interleukins, transformation growth factor beta and congeners of platelet derived growth factor, fibroblast growth factor, epidermal growth factor, hepatocyte scatter factor, leptin, monoclonal antibodies directed against growth factors, modified extracellular matrix components or their receptors, lipid and cholesterol sequestrants, matrix metalloproteases (MMPs), collagenase, plasmin and other agents which may modulate tissue tone, function, and the healing response to organ injury post intervention. Additional examples of such compounds include nitric oxide containing, releasing or producing materials, antiproliferatives as well as antioxidants, a number of which are known.

Hormones, especially reproductive or sex homones, may be particularly advantageous to deliver using these materials. It may also be useful to deliver chemotherapeutics such as BCNU, cisplatin, taxol, Actinomycin D, and other cytotoxic agents. Also addition of stress response inducing agents, evoking heat shock or other mammalian stress protein responses may be desired. Agents include organic and inorganic manganese, tin, cadmium compounds, geldanamycin and analogues oxidizing agents e.g. hydrogen peroxide. Further stress response proteins may also be administered. In certain situations inhibitors of these inducers and of the stress response may also be delivered.

Materials such as attachment peptides (such as the FN cell-binding tetrapeptide Arg-Gly-Asp-Ser (RGDS)), selectin receptors and carbohydrate molecules such as Sialyl Le.sup.x, can be used which serve to attract and bind specific cell types, such as white cells and platelets. Materials such as fibronectin, vimentin, and collagen, can be used to non-specifically bind cell types, to enhance healing. Other proteins known to carry functional RGD sequences include the platelet adhesion proteins fibrinogen, vitronectin and von Willebrand factor, osteopontin, and laminin. Specific RGD peptides are described in U.S. Pat. Nos. 4,517,686 to Ruoslahti, et al., 4,589,881 to Pierschbacher, et al., 5,169,930 to Ruoslahti, et al., 5,149,780 to Plow, et al., 4,578,079 to Ruoslahti, et al., 5,041,380 to Ruoslahti, et al., and Pierschbacher and Ruoslahti, J. Biol. Chem. 262(36), 17294-17298 (1987), Mohri, et al., Amer. J. Hem. 37:14-19 (1991), Aumailley, et al., FEBS 291(1), 50-54 (1991), Gurrath, et al., Eur. J. Biochem. 210, 911-921 (1992), and Scarborough, et al., J. Biol. Chem. 268(2), 1066-1073 (1993). Laminin promotes cell adhesion, migration, differentiation, and growth (Kleinman, et al., J. Cell Biochem. 27:317-325 (1985); Kleinman, et al., Biochem. 25:312-318 (1986); Beck, et al., FASEB J. 4:148-160 (1990). The nonapeptide CDPYIGSR promotes cell attachment and migration (Graf, et al., Cell 48:989-996 (1987), Biochem. 26:6896-6900 (1987)). Further studies have shown that YIGSR-containing peptides can inhibit angiogenesis and tumor metastasis (Grant, et al., Cell 58:933-943 (1989), Iwamoto, et al., Science 238:1132-1134 (1987), Sakamoto, et al., Cancer Res. 51:903-906 (1991). Other peptides include PDSGR and IKVAV. Integrins typically bind to cell adhesion proteins via the rather highly conserved sequence Arg-Gly-Asp X (RGDX), where X is variant depending on the particular cell adhesion protein.

Cells to be incorporated include stromal cells and/or fibroblasts or other mesenchymal cells to facilitate closure of tissue voids. Alternatively glandular epithelial cells, either mature, developing, embryonic/fetal or genetically engineered, may be deposited. These may serve to alter regional or systemic physiology through endocrine or paracrine hormone or other mediator release. Further, neural cells, precursors or tissues may be implanted to facilitate reinnervation and or local adrenergic, cholinergic or other neurotransmitter responses.

In a preferred embodiment, a combination of factors and cells are used to induce angiogenesis in the endomural zone or access tract to the zone. Exemplary angiogenic growth factors include FGF, PDGF, EGF, VEGF, Midkine chemokines, leptins, angiopoeitin, and other growth factors, inflammatory angiogenic polymers or polymer constructs, electoactive or other microinjurious or locally stimulatory polymers. Preferred cells include endothelial cells, EC bone marrow precursor cells, other stems cells smooth muscle cells or precursors, combinations, neural cells or neural stem cells or combinations with above are placed. These are used for example for angiogenesis, myogenesis or myocardial tissue repair in which myocytes—precursor, differentiated, homograft, isograft, allograft or xenograft are placed in the myocardium, with or without polymer adducts or matix protein mixtures, or with neural cells or other adrenerically active or cholinergically active cell types. Means (hard wire or polymer) for electrically driving, pacing, shocking or sensing the neotissue can also be included.

Essentially the same techniques can be used for nerve regeneration or tissue reinnervation by implanting neurons, Schwann cells, astrocytes, glial cells and/or angiogenic precursors. In one embodiment, the nerve cells are administered with polymer matrices, which may include or be formed of bioactive, biodegradable biostable polymers such as polyethyleneglycol polymers, hyaluronic acid, and laminins.

In yet another embodiment, these techniques are used for local endomural delivery of stress response inducing agents or actual stress response proteins. Both physical and chemical stimuli can be used to induce expression of heat shock proteins. The most frequently studied stimuli are heat, oxidants, and heavy metals. Alternatively, or in addition, heat shock proteins can be directly administered to the cells to be treated. Those that are believed to correlate with a response to injury include hsp70, hsp 90 and other cytoplasmic heat shock proteins. Assays to measure the levels of these proteins are well known to those skilled in the art. However, it should be noted that the inducement of heat shock proteins may not be the actual mechanism by which a beneficial effect is obtained, but merely an indicator that appropriate conditions have been used which result in the desired beneficial effect.

Several reviews of heat shock proteins have been published, including Schlesinger, Heat Shock: from bacterial to man (Cold Spring Harbor, Cold Spring Harbor, N.Y. 1982); Lindquist, Ann. Rev. Biochem. 55:1151-1191 (1986); Pelham, H. R. B., Cell 46, 959-61 (1986); Lindquist and Craig, “The heat-shock proteins” Annu. Rev. Genet. 22:631-677 (1988); Pelham, EMBO J. 8:3171-3176 (1989); Schlesinger J. Biol. Chem. 265:12111-12114 (1990); Kaufmann, Immunol. Today 11:129-137 (1990); Morimoto Cancer Cells 3:295-301 (1991); Nover, “HSFs and HSPs—a stressful program on transcription factors and chaperones.” Stress Proteins, and the Heat Shock Response, sponsored by Cold Spring Harbor Laboratory (Cold Spring Harbor, N.Y. USA Apr. 29-May 2, 1991) Nature New Biol. 3:855-859 (1991); and Nover and Scherf “Heat shock protein, in Heat Shock Response (CRC Press, 1991) pp. 41-127.

In most cases, it is possible to physically incorporate the bioactive agent by mixing it with the material prior to application to the tissue surface or within the cavity and polymerization or solidification. The material can be mixed into the monomer solution to form a solution, suspension or dispersion. In another embodiment, the bioactive agent can be encapsulated within delivery devices such as microspheres, microcapsules, liposomes, cell ghosts or psuedovirions, which in themselves affect release rates and uptake by cells such as phagocytic cells.

Bioactive agents can be chemically coupled (conjugated) to the polymeric material, before or at the time of polymerization. Bioactive materials can also be applied to the surface of catheters, trocars, endoscopes, stents or tissue seals or plugs or sensing implants used in the procedures described herein, alone or in combination with the polymeric materials. Catheter and other device or implant bodies are made of standard materials, including metals such as surgical steel and thermoplastic polymers. Occluding balloons may be made from compliant materials such as latex or silicone, or non-compliant materials such as polyethylene terephthalate (PET). The expansible member is preferably made from non-compliant materials such as PET, (PVC), polyethylene or nylon. The balloon catheter portion may optionally be coated with materials such as silicones, polytetrafluoroethylene (PTFE), hydrophilic materials like hydrated hydrogels and other lubricous materials to aid in separation of the polymer coating. Seals and plugs may be made of structural biodegradable or biostable polymers as listed above or from hydrogels polymerized in situ, polymerized ex vivo and transported locally or dessicated hydrogels or organogels or mixtures of the above. Sernsing/telemetring implants may be made of combinations of polymeric and microeletronic, microchip, MEMS or other semiconductor type components.

Several polymeric biocompatible materials are amenable to surface modification in which surface bound bioactive molecules/ligands exhibit cellular binding properties. These methods are described by Tay, Merrill, Salzman and Lindon in Biomaterials 10, 11-15 (1989). Covalent linkages can be formed by reacting the anhydride or acid halide form of an N-protected amino acid, poly(amino acid) (two to ten amino acids), peptide (greater than 10 to 100 amino acids), or protein with a hydroxyl, thiol, or amine group on a polymer. The amine groups on the amino acid or peptide must be protected before forming the acid halide or anhydride, to prevent self-condensation. N-protection is well known by those skilled in the art, and can be accomplished by use of various protecting groups, such as a carbobenzoxy (CBZ) group. The term “protecting group” as used herein refers to a moeity which blocks a functional group from reaction, and which is cleavable when there is no longer a need to protect the functional group. Examples of functional groups include, but are not limited to, amino, hydroxy, thio, and carboxylate groups. Examples of protecting groups are well known to those skilled in the art. A carboxyl-containing compound can contain various functional groups, such as hydroxy, thio, and amino groups, that can react with an acid halide or anhydride. These functional groups must be protected before forming an acid chloride or anhydride to avoid self-condensation. After formation of the acid chloride or anhydride, and subsequent reaction with the hydroxyl, thiol, or amino group(s) on another molecule, the protecting group can be removed in a “deprotecting” step. The N-protected amino groups can be deprotected by means known to those skilled in the art. Any hydroxy or thio groups on these compounds must be protected so as not to react with the acid halides or anhydrides. Examples of suitable protecting groups for alcohols include but are not limited to trialkyl silyl groups, benzyl ethers, and tetrahydropyranyl ethers. These groups can be protected by means known to those skilled in the art, and can be subsequently deprotected after the esterification is complete. Examples of protecting groups can be found in Greene, T. W., and Wuts., P; G. M., “Protective Groups in Organic Synthesis 2d Ed., John Wiley & Sons, Inc., pp. 317-318 (1991), hereby incorporated by reference. A method for preparation of acid halide derivatives is to react the carboxylic acid with thionyl chloride, preferably in benzene or toluene with a catalytic amount of DMF. A known method for producing anhydrides is to react the carboxylic acid with acetic anhydride. In this reaction, as acetic acid is formed, it is distilled out of the reaction vessel. Peptides can be covalently bound to the polymeric material, for example, when the polymeric material is a polymer of an alpha hydroxy acid such as poly(lactic acid), by protecting the amine functionality on the peptide, forming an acid halide or anhydride of the acid portion of the polymer, reacting the acid halide or anhydride with free hydoxy, thiol, or amine groups on the polymer, then deprotecting the amine groups on the peptide to yield polymer having peptide bound thereto via esterification, thioesterification, or amidation. The peptide can also be bound to the polymer via a free amine using reductive amination with a dialdehyde such as glutaraldehyde. The ester groups on a polyester surface can be hydrolyzed to give active hydroxy and carboxyl groups. These groups can be used to couple bioactive molecules. Preferably, before converting the active carboxylate group to the acid halide or anhydride form, the active hydroxy group is protected to avoid reaction with the resulting acid halide or anhydride. As a non-limiting example, the active hydroxy group can be protected as a benzyl ether. The active carboxyl group can then be converted to the acid halide or anhydride, and reacted with a hydroxy or amino group on a second compound to form an ester or amide linkage. The O-protected hydroxy group can then be deprotected.

Coupling agents such as carbodiimides, diisocyanates, or organosilanes can be used to bind polymers, or metals and ceramics to bioactive agents covalently. For example, a metal stent may be treated with an aqueous solution of an aminotrialkoxy silane. These form an amino functional surface which can react with carboxy-functional proteins, for durable attachment or controlled release. Carbodiimides can react with carboxyl functional groups to produce amino-reactive intermediates. Carboxy functional polymers can be reacted to form N-hydroxy succinimide esters which are very reactive with amino groups on peptides. This chemistry has been used to form surgical sealants PEG-di-N-hydroxysuccinimide and albumin, Barrows, et al., 3M Corporation, but could be used to couple bioactive molecules to polymers.

2. Application of Polymeric Materials

In general terms, the polymeric material is a biocompatible polymeric material having a variable degree of fluency in response to a stimulus or mechanical pressure, as described above. The material is such that it is substantially non-fluent in vivo upon completion of the coating process. The material, in its fluent form or a conformable form, is positioned in contact with a tissue or device surface to be coated and then stimulated to render it non-fluent or conformed, as described above. The polymeric material is applied to the cavity or endomural void using catheters, syringes, or sprays, depending on the tissue surface or device to which it is applied, using the devices described above or devices known to those skilled in the art.

The coating typically will be applied to a tissue surface such as the media of an artery, the urethra, brain or the myocardium using some type of catheter, trocar or scope. The coating material is preferably applied using a single catheter or similar device with single or multiple lumens. The catheter should be of relatively low cross-sectional area. A long thin tubular catheter manipulated using endoscopic guidance is preferred for providing access to the interior of organ areas. Alternatively the device may have direct vision capabilities via contained fiberoptics or actual tip cameras (CCD, C-MOS,etc) or via echo sensing, US sensing or GPS positioning systems.

Application of the coating material may be accomplished by extruding a solution, dispersion, or suspension of monomers, polymers, macromers, or combinations thereof through a catheter to coat or fill a tissue surface or cavity, then controlling formation of the coating by introducing crosslinking agents, gelling agents or crosslinking catalysts together with the fluent material and then altering the conditions such that crosslinking and/or gelling occurs. Thus, when a balloon catheter is used, a flow of heated or chilled fluid into the balloon can alter the local temperature to a level at which gelling or cross-linking is induced, thereby rendering the material non-fluent. Localized heating or cooling can be enhanced by providing a flow of heated or chilled liquid directly onto the treatment site. Thermal control can also be provided, however, using a fluid flow through or into the balloon, or using a partially perforated balloon such that temperature control fluid passes through the balloon into the lumen. Thermal control can also be provided using electrical resistance heating via a wire running along the length of the catheter body in contact with resistive heating elements. This type of heating element can make use of DC or radio frequency (RF) current or external RF or microwave radiation. Other methods of achieving temperature control can also be used, including light-induced heating using an internal optical fiber (naked or lensed). Altrnatively as self-contained fluid flow system allowing inflow and outflow of fluids to the ballon, actuator or other material applying tip of surface may control polymer flow, melt, setup and cooling and fixation. The polymer formulation can contain components which convert light into heat energy. Similar devices can be used for application of light, ultrasound, or irradiation.

Alternatively the polymers may be delivered as solid materials of various configurations e.g. rods, speheres, folded sheets, yarms, meshes, twines, ropes, particles, amorphous shapes, flakes, etc. Similarly hydrogel materials may be dlivered with the above physical geometries in either the hydrated, partially hydraed or dessicated form. Further defined hydrogel shapes shuch as spikes, spheres with wicks and other tract+void shapes may be delivered for the purpose of void sealing or plugging or repair.

Any of the foregoing materials can be mixed with other materials to improve their physiological compatibility. These materials include buffers, physiological salts, conventional thickeners or viscosity modifying agents, fillers such as silica and cellulosics, and other known additives of similar function, depending on the specific tissue to which the material is to be applied.

The process of fixing the shape of the polymeric material can be accomplished in several ways, depending on the character of the original polymeric material. For example, a partially polymerized material can be expanded using a balloon after which the conditions are adjusted such that polymerization can be completed, e.g., by increasing the local temperature or providing UV or visible radiation through an optical fiber. A temperature increase might also be used to soften a fully polymerized sleeve to allow expansion and facile reconfiguration and local molding, after which it would “freeze” in the expanded position when the head source is removed. Of course, if the polymeric sleeve is a plastic material which will permanently deform upon stretching (e.g., polyethylene, polyethylene terephthalate, nylon or polyvinyl chloride), no special fixation procedure is required.

The present invention will be further understood by reference to the following non-limiting examples.

### Example 1

### Application of Tissue Adhesive in a Cavity.

An incision in an organ is made. A tissue adhesive is then applied within the cavity to enhance healing of the wound. The following are examples of useful tissue adhesives to close the voids.

a. 1 gm of 50 mg Fibrinogen/ml is mixed in situ with 0.3 g of 150 NIH U thrombin/ml containing 100 mM CaCl₂ at the site of the cavity. This forms a tissue glue within 90 sec.

b. 2 gm of 100 mg Fibrinogen/ml is mixed in situ with 0.3 g of 150 NIH U thrombin/ml containing 100 mM CaCl₂ at the site of the cavity. This forms a tissue glue within 30 sec.

c. 1 gm of 50 mg Fibrinogen/ml is supplemented with 2500 kIU Aprotinin/ml with 12.5 mg epsilon-aminocaproic acid/ml. The solution is mixed in situ with 0.3 g of 150 NIH U thrombin/ml containing 100 mM CaCl₂ at the site of the cavity. This will delay the in vivo degradation of Fibrin glue and retain the collapsed state of the cavity for a longer duration of time. Tranexamic acid can be used instead of aprotinin for better healing response of the tissue.

### Example 2

### Dehydration of Tissue Before Application of Glue.

In another example, a cavity is aspirated following washing with a concentrated ethanol solution (80% w/w in water). This process dehydrates the local area of the cavity. The in situ Fibrin glue is applied as described above to promote better adhesion of the tissue.

Modifications and variations of the methods and compositions described above will be obvious to those skilled in the art and are intended to be encompassed by the following claims.

We claim:

1. A method of treatment comprising locally penetrating and entering the body of an organ, organ component or tissue structure with minimal damage to obtain access to endomural zones of an organ.

2. The method of claim 1 further comprising depositing in the midzone therapeutic agents and systems.

3. The method of claim 2 wherein the therapeutic agents are selected from the group consisting of drugs, cells and polymers and diagnostic and/or therapeutic devices.

4. The method of claim 3 wherein the polymers may be degradable or non degradable.

5. The method of claim 3 wherein the polymers are selected from the group consisting of solid matrices, porous matrices, hydrogels, organogels, colloidal syspensions, microparticles and microcapsules, anoparticles and combinations therof.

6. The method of claim 3 wherein the drugs are selected from the group consisting of anti-infectives, antibiotics, antifungal, antihelminthic, antiparasistic agents, anticancer agents, anti-proliferative agents, anti-migratory agents, anti-inflammatory agents, metalloproteases, proteases, thromblytic agents, fibrinolytic agents, steroids, hormones, vitamins, charbohydrates, lipids proteins, peptides and enzymes.

7. The method of claim 3 wherein the drugs are proliferative growth factors selected from the group consisting of PDGF, FGF, TGF, EDGF, Epidermal GF, NGF, ILGF, Hepatocyte scatter factor, angiogenic growth factors, serum factors, collagen, laminin, tenascin, SPARC, thrombospondin, fibronectin, vimentin and other matrix factors.

8. The method of claim 3 wherein the cells are selected from the group consisting of autogenous similar cells (i.e. mesenchymal for mesenchymal) from adjacent normal zones of the same or different organs.

9. The method of claim 3 wherein the cells are selected from the group consisting of autogenous differing cells (i.e. mesenchymal for ectodermal or splenocytes for endothelial cells) from adjacent normal zones of the same or different organs.

10. The method of claim 3 wherein the cells are therapeutic factors produced by or in the form of stem cells or other progeneitor cells.

11. The method of claim 3 wherein the cells are explanted and clonally or otherwise expanded in vitro for implantation, either without genetic modification or genetically modified, before implantation.

12. The method of claim 3 wherein the therapeutic factors are selected from the group consisting of genes, plasmids, episomes, viruses, viroids, or other microorganisms for therapeutic or synthetic purpose.

13. The method of claim 3 wherein the therapeutic factors are heat shock or stress response proteins or inducers of heat shock or stress response proteins.

14. The method of claim 1 further comprising where a cavity or containment space or reservoir area does not exist in the endomural zone, creating such a space for therapeutic placement.

15. A device comprising a hollow tubular member with an end penetrating or cuting means causing minimal collateral damage and means for delivery of therapeutic agents into endomural tissue.

16. The device of claim 15 wherein the member is rigid made of metal, polymer, or composite.

17. The device of claim 15 wherein the member is flexible and comprises a catheter-like device.

18. The device of claim 15 wherein the member is attached to a single or multiple reservoirs for therapeutic agent containment and delivery.

19. The device of claim 15 wherein the member has an expansile cutter at the distal end to create a tissue space.

20. The device of claim 15 further comprising diagnostic or therapeutic sensors.

21. The device of claim 15 further comprising projectile means to ballistically transfer particles through the ectoluminal or endoluminal zone for retention in the endomural zone.

22. The device of claim 21 wherein the projectile means is selected from the group comprising mechanical acceleration, electrical transfer, spark explosion, and gas explosion.

23. The device of claim 15 further comprising means for indirect or direct guidance means.

24. The device of claim 23 wherein the means for direct guidance is selected from the group consisting of fiber optic imaging systems, endoscopes, direct tip cameras, CCD, C-MOS or other chip or electrical video systems, ultrasound or GPS positioning systems.

25. The device of claim 15 in a kit comprising a void filling material which contains electroactive agents.

26. The device of claim 15 comprising a void filling material or implant which can locally sense, store or telemeter physical, chemical or biological information.

27. The device of claim 15 comprising electoactive or electroconductive polymers which may be directly or externally activated via transcutaneous energy delivery to elicit positive or negative galvanotaxis (tissue healing or cell movement to or from based on local persistent or intermittent electrical current).

28. The device of claim 15 comprising a therapeutic for induction of angiogenesis or myogenesis.

29. The device of claim 28 comprising a therapeutic selected from the group of angiogenic growth factors, inflammatory angiogenic polymers or polymer constructs, electoactive or other microinjurious or locally stimulatory polymers.

30. The device of claim 28 comprising cells selected from the group consisting of endothelial cells, EC bone marrow precursor cells, other stems cells smooth muscle cells or precursors, combinations, neural cells or neural stem cells or combinations with above are placed.

31. The device of claim 15 for nerve regeneration.

32. The device of claim 15 comprising a bioactive polymer.

33. The device of claim 15 comprising stress response inducing agents or actual stress response proteins.
